(19) United States
(12) Patent Application Publication
(10) Pub. No.: US 2006/0293634 A1
(43) Pub. Date: Dec. 28, 2006

(54) Method of promoting relationship marketing with a consumer of a personal care article

(75) Inventors: Brain Lee Thomas, Neenah, WI (US); Shelley Rae Frost, Suamico, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/166,463

(22) Filed: Jun. 23, 2005

(51) Int. Cl. A61F 13/15

(52) U.S. Cl. 604/385.01

(57) ABSTRACT

A method for providing an auxiliary benefit to a consumer includes providing a personal care article (22) which has a liquid-permeable topsheet layer (36), a backsheet layer (38) operatively connected with the topsheet layer (36), and a removable layer (42) which is releasably connected to the personal care article (22). A redemption certificate (44) is provided on the removable layer (42). Also provided are conversion instructions for employing the redemption certificate (44) to obtain a separately provided item of perceived, additional benefit. The item of benefit is provided separate from the personal care article (22).

### BACKGROUND OF THE INVENTION

The present invention relates generally to personal care articles and more particularly to a technique which can more effectively establish and nurture a business relationship between a seller of the articles and a consumer of the articles.

The personal care articles can, for example, be absorbent articles, and such articles may be feminine care articles, such as panty liners and feminine napkins and the like. Desired groupings of the articles have been packaged using conventional techniques and outer containers, such as pouches, bags, boxes, cartons and the like.

The individual products have included areas coated with adhesive materials for attaching the product to a crotch area of clothing to hold the product in place adjacent a wearer's pudendum during use. Typically, a label or covering layer has been removably attached to the adhesive coating to cover the coating and to prevent attachment of the product before the user removes the covering from the adhesive coating. In the past, messages related to instructions for using the product and/or designations of a source of the product (e.g., a trademark) have been printed on the removable covering layers.

Other items of information that are beneficial to the consumers have been provided on the covering that has been placed over the adhesive coating. The items of beneficial information have also been located on the wrappers or pouches employed to enclose the individual products. Such information has included instructions and information pertaining to the source of the goods (e.g. trademarks and tradenames). Messages which provide information regarding previously identified needs and desires of the consumers have been disclosed in U.S. Patent Application Publication 2003/0088224 entitled ABSORBENT PRODUCT HAVING MESSAGE by Ceman et al.

In addition, promotional coupons have been provided on or within the outer packaging of consumer products. Such distributions of the coupons, however, have been expensive, and have experienced reduced effectiveness, particularly with younger consumers who have a preference for computerized or other electronic communications. Additionally, the positioning of coupons on the outer packaging has not been desirable or permitted in various circumstances. As a result, there has been a continuing need for improved techniques for attracting the attention and loyalty of consumers.

### SUMMARY OF THE INVENTION

A method for providing an auxiliary benefit to a consumer includes providing a personal care article which has a liquid-permeable topsheet layer, a backsheet layer operatively connected with the topsheet layer, and a removable layer which is releasably connected to the personal care article. A redemption certificate is provided on the removable layer. Also provided are conversion instructions for employing the redemption certificate to obtain a separately provided item of perceived, additional benefit. The item of benefit is provided separate from the personal care article.

By incorporating its various aspects and features, the method of the present invention can provide an improved and more effective system for attracting and maintaining the loyalty of consumers. The system can provide enhanced effectiveness, particularly with younger consumers who have a preference for computerized or other electronic communications.

### BRIEF DESCRIPTION OF THE DRAWINGS

The invention will be more fully understood and further advantages will become apparent when reference is made to the following detailed description of the invention and to the drawings in which:

FIG. 1 shows a plan view of the bodyside of a representative article which can be employed with the present invention.

FIG. 2 shows a plan view of the garment-side of a representative article which can be employed with the present invention.

FIG. 3 shows a plan view of a representative article which can be employed with the present invention, where the individual article includes a corresponding, individual envelope or wrapper.

FIG. 4 shows an elevational view of a package that contains a plurality of individually wrapped articles.

FIG. 5 shows a representative flow diagram which provides further details of the invention.

Corresponding reference characters indicate corresponding parts throughout the several views of the drawings.

### DETAILED DESCRIPTION OF THE INVENTION

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements. Such terms are intended to be open-ended terms that specify the presence of any stated features, elements, integers, steps, or components, and are not intended to preclude the presence or addition of one or more other features, elements, integers, steps, components, or groups thereof.

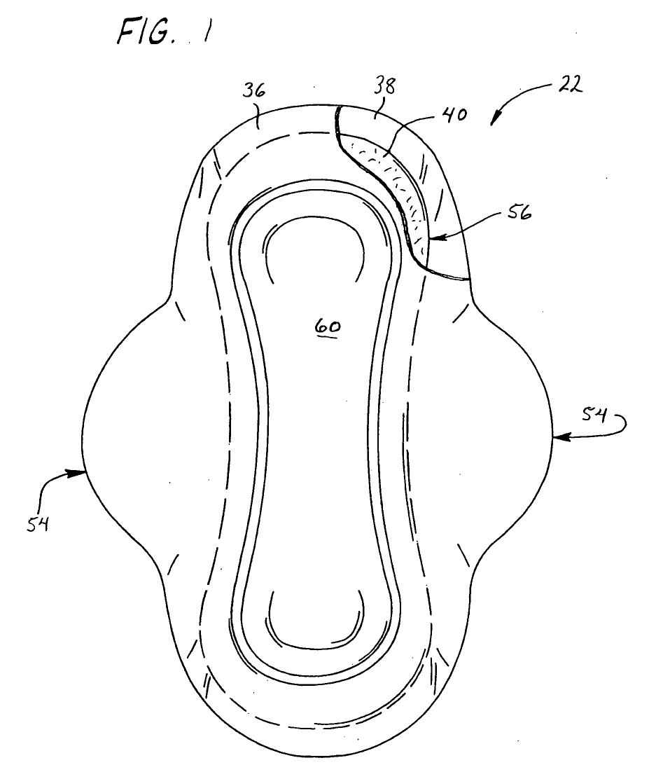

As representatively shown in FIGS. 1 through 5, a method for providing an auxiliary benefit to a consumer includes providing a personal care article 22 which has a liquid-permeable topsheet layer 36, a backsheet layer 38 operatively connected with the topsheet layer 36, and a removable layer 42 which is releasably connected to the personal care article 22. A redemption certificate 44 is provided on the removable layer 42. Also provided are conversion instructions for employing the redemption certificate 44 to obtain a separately delivered item of perceived, additional or auxiliary benefit. In a desired aspect, the item of benefit can be provided and delivered in a manner that is spatially and/or temporally separated from the delivery of the personal care article 22.

An individual removable layer 42 can be operatively joined to each of the articles 22, and the removable layer is intended to be selectively repositioned relative to the article prior to using the article, to thereby enable a full functionality of the article. For example, the position of the removable layer 42 can be selectively moved or otherwise changed relative to the article, or the removable layer can be removed from the article. With respect to a predetermined plurality of individual articles 22, a redemption certificate 44 may or may not be located on each of the selected removable layers 42 associated with the individual article. A portion of the redemption certificate 44 may be configured to communicate a selected message to the user or consumer. The message can be related to a product message, a benefit communication, an incentive message or the like, as well as combinations thereof.

In a desired feature, the selective repositioning of the removable layer prior to using the article causes a corresponding, selective repositioning of at least a significant portion of the redemption certificate. Particular arrangements can provide a corresponding, selective repositioning of substantially all of the redemption certificate.

By incorporating its various aspects and features, the present invention can provide an improved and more effective system for providing auxiliary benefits to a consumer. The method of the invention can more effectively communicate messages that are intended to attract and maintain the loyalty of consumers, and can provide enhanced effectiveness, particularly with younger consumers who have a preference for computer based communications or for other types of electronic communications. The communications can be better targeted to selected audiences and to consumers having selected levels of interest. The intended communications with the consumer can be conducted in greater privacy, and can be readily continued with modern technologies, such as electronic, e-mail and other types of internet-based communications.

In a particular configuration of the invention, the article 22 can be a personal care article, and in a further configuration, the article can be absorbent articles. Additionally, the article may be disposable. As used herein, the term “absorbent article” refers to devices which can absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats. In desired arrangements, the method of the invention can be employed with feminine care articles.

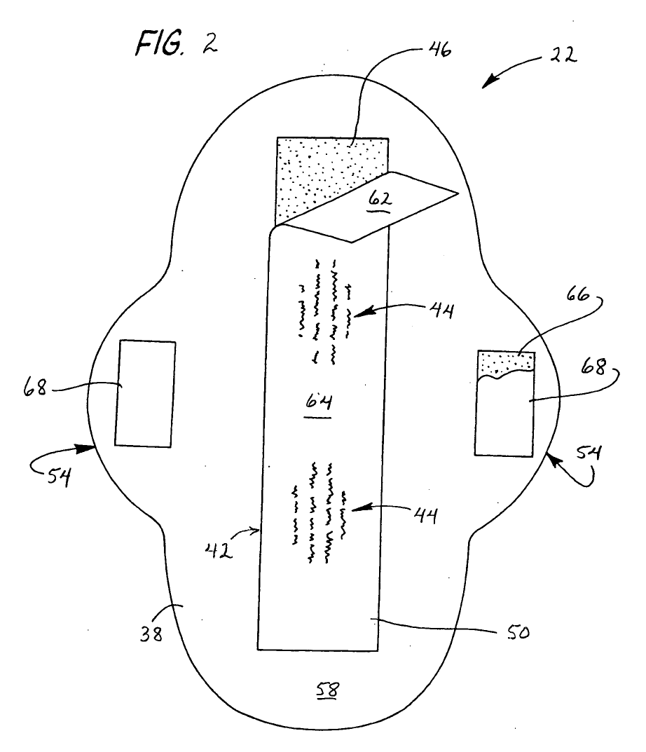

Referring now to the drawings and in particular to FIGS. 1-3, a representative article is designated in its entirety by the reference numeral 22. Although the example article 22 shown in FIGS. 1-2 is an absorbent feminine napkin, those skilled in the art will appreciate that the present invention may be employed with other feminine care articles, such as panty liners, as well as other personal care absorbent articles, such as adult care incontinence products, child care products and infant care products.

As representatively shown, the personal care article 22 can have a laminated structure which includes a liquid-permeable topsheet layer 36, and a backsheet layer 38. In particular aspects, the article can further include an absorbent body 40 that is positioned and held between the topsheet layer and backsheet layer. The topsheet layer 36 can comprise any operative, liquid-permeable material. For example, the topsheet layer can include a polymer film, a woven fabric, a nonwoven fabric or the like, as well as combinations thereof. The employed polymer films may be porous, or may be treated or otherwise processed to impart the desired level of liquid-permeability.

The backsheet layer 38 can comprise a polymer film, a woven fabric, a nonwoven fabric or the like, as well as combinations thereof. In desired arrangements, the backsheet layer 38 can be configured to be operatively liquid-impermeable, and can sufficiently block the movement of body-liquids through the thickness of the backsheet layer during ordinary use. In another feature, the backsheet layer 38 can be configured to be gas-permeable or “breathable”. Such breathable backsheet layer materials are well known and available from commercial vendors.

The personal care article 22 can further include an absorbent body 40 which is operatively sandwiched between the topsheet layer 36 and the backsheet layer 38. The absorbent body 40 is capable of operatively absorbing a hygienically effective amount of urine or menses. It should be appreciated that the article 22 may or may not include the absorbent body 40. When present in the article, the absorbent body 40 can include any operative absorbent material. Examples of suitable absorbent materials can include natural fibers, synthetic fibers, woodpulp fibers, cellulosic fibers, synthetic polymer fibers, thermoplastic binder fibers, bicomponent binder fibers or the like, as well as combinations thereof. Additionally, the absorbent body 40 can include superabsorbent materials which can typically absorb and retain large amounts of aqueous liquids per unit weight of the superabsorbent material. The superabsorbent materials have very high absorbent capacities and swell to form hydrogels that are substantially water-insoluble. Such superabsorbent materials are well known and are readily available from commercial vendors.

Particular arrangements of the absorbent body 40 in a feminine care product can be configured to provide a discrete amount of absorbent saturation capacity. In particular arrangements, the saturation capacity can be a minimum of about 0.1 grams of menses simulant, or less. In other aspects, the absorbent body has a saturation capacity which can be up to a maximum of about 100 grams of menses simulant.

A suitable menses simulant for determining absorbent capacity is composed of swine blood diluted with swine plasma to provide a hematocrit level of 35% (by volume). A suitable device for determining the hematocrit level is a HEMATOSTAT-2 system, available from Separation Technology, Inc., a business having offices located in Altamonte Springs, Fla., U.S.A. Alternatively, a substantially equivalent device or system may be employed.

The absorbent body has a peripheral edge 56. Additionally, the article 22 can have a first, garment-facing side 58 (e.g. FIG. 2), and a second, bodyside 60 that is located opposite the garment-side 58. Tabs or side-panels, generally designated by 54, may be incorporated, and an individual side-panel can be configured to extend laterally from each lateral edge of the absorbent body 40 for wrapping around the crotch of the user's underwear (not shown) to protect it from staining. The tabs may also be referred to as “wings”. As illustrated in FIG. 2, each side-panel 54 may include a tab fastener, such as provided by the representatively shown adhesive strip 66 or other adhesive region, for attaching either or both side-panels in a conventional manner to an outside surface of a crotch section of the user's underwear to hold the side-panels 54 of the personal care article 22 in place around the underwear during use. The adhesive strips 66 may be covered with a removable peel strip member 68 to prevent the adhesive strips from sticking to other surfaces until ready for use.

As illustrated, a garment-attachment mechanism, such as provided by the representatively shown region of garment adhesive 46, can be also applied to the garment side of the article, thereby permitting a user to attach the article to a preselected surface, such as the inside surface of the crotch of the user's underwear, to hold the personal care article 22 in place on the underwear during use. In a particular aspect, the garment-attachment mechanism 46 can be operatively connected to a garment-side of the backsheet layer 38. In another aspect, the removable layer 42 can be a peel-strip layer that has been operatively attached in a storage position to overly and cover the garment-attachment mechanism 46. In a desired arrangement, the garment-attachment mechanism 46 can be a garment-adhesive.

As representatively shown, the garment-attachment mechanism 46 can include a coating or other distributed pattern of adhesive (e.g., a two-sided adhesive film or tape). The garment adhesive can be disposed on the garment-side of the backsheet 38, and can be configured to provide an operative garment-fastener. Accordingly, each individual article can include a garment adhesive layer that is secured to an outward, garment-facing surface of the corresponding backsheet layer 38 of the individual article. The removable layer 42 of each article 22 may have the configuration of an article-layer 50 (e.g. a peel-strip layer) that is removably secured to the garment-adhesive layer or other garment-attachment mechanism of each article 22.

The garment-fastener 46 may have any operative size and shape without departing from the scope of the present invention. In a particular embodiment, for example the adhesive garment-fastener can be generally rectangular, and can have a width of about three centimeters and a length of about 17 centimeters.

The label, peel-strip or other removable article-layer 50 can be releasably attached to the garment-attachment mechanism. For example, the removable layer can have a configuration that is releasably joined to cover the adhesive fastener 46 and prevent premature attachment of the article 22 before the user separates the removable layer from the adhesive coating. Although the removable article-layer 50 may have any operative size and shape without departing from the scope of the present invention, a desired arrangement of the article-layer 50 can have a size and shape that is selected to entirely cover the adhesive fastener 46 when the article-layer is attached to the adhesive coating. More particularly, in one embodiment, the removable article-layer 50 can be rectangular and can have a width of about four centimeters and a length of about 17 centimeters. Because the removable article-layer 50 can be wider than the adhesive fastener 46, the removable layer can present a loose margin which can be grasped easily by the user when removing the covering, article-layer 50 from the garment-fastener adhesive 46.

It should be readily appreciated that the selected tab fastener (e.g. adhesive fastener 66) and/or the selected garment-attachment fastener (e.g. adhesive fastener 46) can be provided by any operative fastening device or system. For example, the fastener mechanism or system can include an adhesive, a cohesive, an interengaging mechanical fastener, a cooperative component of a hook-and-loop fastener, a magnetic fastener, an electrostatic fastener or the like, as well as combinations thereof.

As representatively shown, the garment-attachment fastener can be an operative garment-adhesive, and the removable article-layer 50 can have a release side 62 for contacting the adhesive fastener 46 when the removable layer is attached to the adhesive. Additionally, the removable layer 42 can have an outward-facing side 64 positioned opposite the release side. As will be explained in greater detail below, the outward side 64 of the removable layer 42 can include a redemption certificate, generally designated by 44, thereon.

In a particular arrangement, the corresponding redemption certificate 44 of each article 22 may be located on an exposed surface of the removable article-layer 50, which is arranged to face outwardly toward the user. As representatively shown, for example, the redemption certificate 44 can be located on an outward-side surface 64 of the removable article-layer. Alternatively, the redemption certificate 44 may be positioned on an inward, body-facing surface 62 of the removable article-layer 50, and the removable layer may or may not be configured to be sufficiently transparent to allow an operative viewing of the redemption certificate through the removable article layer.

An optional arrangement may include a wing peel-strip layer which is provided by an existing, already present layer of the material employed to form the tabs or side flap “wings” 54. In a particular aspect, the tabs can have a storage position in which at least one, and desirably both, of the tabs 54 are positioned against the outward-facing, garment-side surface of the backsheet layer 38. Additionally, the desired redemption certificate 44 can be located on a surface of the tab 54 in a manner that allows the redemption certificate to be at least partially visible to the user when the tab is in its storage position and the fresh, unused article is presented to the user from said opening mechanism. For example, either or both of the wing tabs 54 may be folded and positioned generally adjacent and against the outward garment-facing surface of the backsheet layer 38 to provide the desired storage position. The redemption certificate can then be located on a surface of the tab that is outwardly-facing when the tab is in its storage position. From its storage position, the tab with the redemption certificate is intended to be moved or otherwise repositioned prior to using the article, to enable full functionality of the article. In particular, the tabs can be moved and extended to laterally outboard positions to allow a positioning and securing of the tabs around the lateral side edges of the crotch region of the user's undergarment. Thus, the article can be located and operatively reconfigured in the undergarment prior to using the article, and the reconfigured article will allow a desired, full functionality.

In the various arrangements of the invention, the redemption certificate 44 may be applied to the selected removable layer 42 by employing any operative technique (e.g., embossing) without departing from the scope of the present invention. As representatively shown, the redemption certificate can be printed on the removable layer using conventional printing techniques.

It should be readily appreciated that the personal care article 22 can include various conventional structures, and such structures are well known by those skilled in the art. Thus, the materials used in making the personal care article 22 described above, the detailed construction of the personal care article, and the method of manufacturing the personal care article are well known and will not be described in further detail. Examples of conventional personal care article articles 22 are described in U.S. Pat. No. 5,429,630, which was issued Jul. 4, 1995.

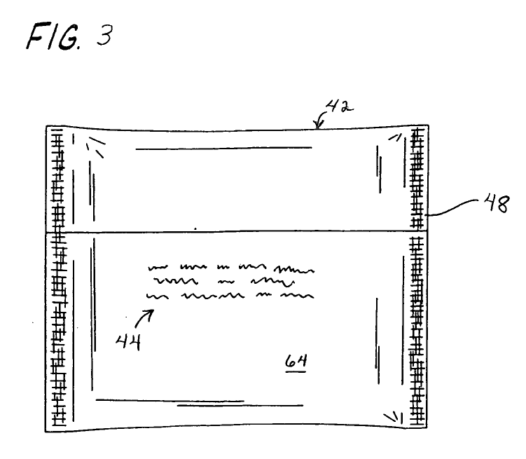

The article 22 may be folded and individually packaged in various ways without departing from the scope of the present invention. As representatively shown in FIG. 3, the personal care article may be folded and placed in an individual envelope or wrapper, or in other operative individual packaging. In a particular feature, the corresponding removable layer 42 of each article 22 can be configured to provide a wrapping layer 48 for an individual article, and the wrapping layer can extend across at least two major surfaces of its corresponding article 22 and can at least partially enclose the individual article. Accordingly, an individual article 22 can have an appointed removable layer which includes its corresponding, individual wrapper layer 48.

As representatively shown, the redemption certificate 44 may be integrally formed with the wrapping layer. In an alternative arrangement, the redemption certificate 44 may include a component that is provided separate from the wrapping layer 48, and is subsequently attached or otherwise assembled to the wrapping layer.

The wrapping layer may or may not be formed from a material that has sufficient transparency to allow an operative viewing and reading of the article 22 by the user through the wrapping layer material. The wrapping layer 48 can, for example, be configured to provide an envelope or pouch which encloses its corresponding article 22. The wrapping layer 48 can comprise any operative material. For example, the wrapping layer can include a polymer film, a woven fabric, a nonwoven fabric, a composite laminate or the like, as well as combinations thereof.

The redemption certificate 44 on the selected removable layer 42 (e.g. article-layer 50, wrapping layer 48) can be directed to communicating a desired message to the user. The message can be related to a product message, a benefit communication, an incentive message or the like, as well as combinations thereof. Further, the message can be intended to establish and/or nurture a business relationship between a seller of the absorbent articles and a consumer. For example, the redemption certificate 44 may provide a communication pertaining to a contest, a prize, a sweepstakes, a code or registration number for use with a computer internet website or the like, as well as combinations thereof.

The appointed removable layer 42 (e.g. article-layer 50 and/or wrapper 48) may optionally include messages related to the personal use of the absorbent article, or messages related to a designating of a source of the absorbent article. Desirably, the removable layer can be configured to be substantially free of messages that are related to the personal use of the article 22, or of messages that are related to the designating of the source of the personal care article (e.g., a manufacturer's trademark).

In a desired feature, the article can be configured such that the movement, removal or other change in relative position of the removable layer 42 causes a corresponding movement, removal or other change in relative position of the redemption certificate 44. When the position of the removable layer is operatively changed to enable the functionality of the article, the position of the entire redemption certificate or the position of a significant portion of the redemption certificate is modified by the positional change of the removable layer 42. As will be appreciated by those skilled in the art, it may be desirable to partially or completely remove the removable layer 42 from the personal care article 22 to apply and operatively join the personal care article to the wearer's underwear or other undergarments and thereby to enable a substantially full functionality of the personal care article. Thus, the user can more immediately come in an operational, communicative contact with the removable layer 42, and the user's attention can be more effectively drawn to the message. The configurations of the present invention can be particularly beneficial when relaying messages such as those related to a product benefit or a purchasing incentive.

In a particular aspect, the redemption certificate 44 can include a coupon which is redeemable for a predetermined monetary amount. In a desired aspect, the redemption certificate 44 can include an authentication code. Any operative of an authentication code may be employed. For example, the authentication code can include a bar code, a number code, a text code, an alpha-numeric code or the like, as well as combinations thereof. In a desired aspect, the authentication code can be machine-readable.

The redemption certificate 44 can include a predetermined set of text printed on the peel-strip layer 42. Alternatively, the redemption certificate 44 can include a distributed pattern of perforations, a distributed pattern of embossments or the like, as well as combinations thereof.

In another aspect, the redemption certificate 44 can be configured to be substantially, non-recognizable to ordinary human vision when the removable layer 42 is in its storage position, and is configured to become operatively recognizable to ordinary human vision upon a removal of the removable layer 42 from the article 22. The change in the ability to recognize the redemption certificate 44 can be provided by any operative structure or technique. For example, the redemption certificate 44 can be configured to become operatively recognizable to ordinary human vision upon a removal of the peel-strip layer from the garment-attachment mechanism 46. In a particular configuration, the redemption certificate 44 can be located on a body side surface of the peel-strip layer.

As another example, the change in the ability to recognize the redemption certificate can include a code or text provided by an array of perforations where perforations and the associated code or text becomes more readily discernable after the redemption certificate has been removed from the article. In a further example, the redemption certificate can include a code or text provided by a color-shifting ink or an ink that becomes activated and visible to human sight when the ink has been exposed to selected wavelengths of light. Still another example of such change in the recognition ability can include a code or text which has been provided by a pattern of ink, adhesive or other coating, and which subsequently becomes revealed after the redemption certificate has been removed from the article.

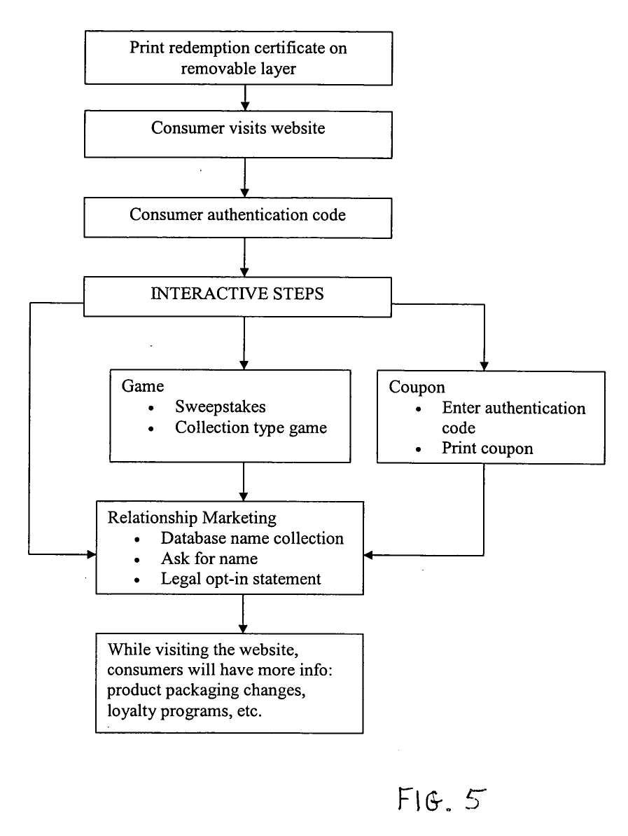

With reference to FIG. 5, the method of the invention can further include a providing of instructions to initiate an interactive contact with a benefit provider. In a particular feature, the interactive contact can include an internet web-based, computerized contact. In a desired aspect, the interactive contact can include instructions that have been operatively positioned on the removable layer 42 (e.g. the peel-strip, article-layer 50). Optionally, the interactive contact can include instructions that have been operatively located on another component of the article. The other component of the article may, for example, be the backsheet layer 38, the wrapping layer 48, a pouch or other form of packaging 20.

Another aspect of the invention can include authorizing the establishment of an interactive communication with a person seeking to redeem or actualize the redemption certificate 44. The interactive communication can include an oral communication, a manual communication, a written text communication, a computerized communication or the like, as well as combinations thereof. A suitable computerized communication can employ a mobile, cellular telephone; an electronic, personal data assistant; an electronic computer configured with an operative user-interface, such as a graphical user interface; or the like.

The method of the invention may include an authorizing of the providing of a coupon in response to a receipt of the authentication code. In a desired feature, the coupon can be machine-printable by a person who has provided the authentication code, and the coupon may be redeemable for a predetermined monetary amount. Such authorizing can be accomplished by various means. Suitable means can include employing contractual arrangements, systems, processes and computer software that are well known in the art. For example, such authorizing systems can include proprietary or purchased software that generates a master list containing codes, and incentive levels that match those codes. Such systems are well known and available from commercial vendors. For example, a suitable system is available from Coupons, Inc., a business having offices located in Atlanta, Ga., U.S.A.

A further aspect of the invention can include authorizing the providing of a game-entry in response to a receipt of the authentication code. The game-entry may, for example, include an entry to a sweepstakes game, a collection game or the like, as well as combinations thereof. Various sweepstakes games may be employed. For example, a code number and/or an instant-win message can be included on the redemption certificate. With the code number, the consumer can check (e.g. via an Internet site) to see if the consumer has won a prize or other item of perceived, consumer-value. A redemption certificate may optionally communicate an “instant” win, entitling the consumer to a free consumer product of the same size and brand.

Various collection games may be employed. For example, the collection game may include a series on game pieces needed to complete a sequence, or to complete a predetermined array. More particular examples may include MONOPOLY game pieces; collections of baseball, basketball and football players; collections of other sports players; collections of race car drivers; collections of celebrities; or the like. For example, a collection game piece can be included on the redemption certificate, and a consumer can collect a certain number and/or type of game pieces to obtain a prize or other item of value. In another example, the redemption certificate may include clues used to win a prize or other item of consumer-value. Consumers may use the clues to solve or provide the answer to a puzzle or question. Where there are multiple submittals of the correct answer, the submittals may be operatively recorded and then entered into a drawing for the final prize.

As another example, a television or other broadcast show may run a promotion which requires a person to view the show to collect clues used to win a prize. The consumer would obtain a series of words and/or graphics provided during an individual broadcast or series of broadcasts, and then attempt to “match” or recreate a winning-sequence using words and/or graphics found on the redemption certificate that have been printed or otherwise provided on the product being promoted. Where there are multiple submittals of the winning-sequence, the submittals may be operatively recorded and then entered into a drawing for the final prize.

In a particular aspect, the method of the invention can include authorizing the collection and/or verification of information, such as marketing information, in response to a receipt of the authentication code. In another aspect, the method of the invention can include authorizing the presentation of an interactive, multi-pictorial graphical display in response to a receipt of the authentication code. Still another aspect can include authorizing the presentation of an interactive, multi-media motion picture display in response to a receipt of the authentication code. Still a further aspect of the invention can include authorizing the providing of a consumer-loyalty benefit in response to a receipt of the authentication code. Examples of a suitable consumer-loyalty benefit can, for example, include a purchase incentive, such as price discount or the availability of an additional item at little or not additional cost.

In the various arrangements and configurations of the invention, the authorizing can be accomplished by employing contractual arrangements, systems, processes and/or computer software that are well known in the art.

An effective authorization can operatively be provided by a producer or distributor of the personal care article 22. Alternatively, an operative authorization can be provided by at least one intermediate entity or intermediary that has been designated or appointed by the manufacturer or producer of the personal care article 22.

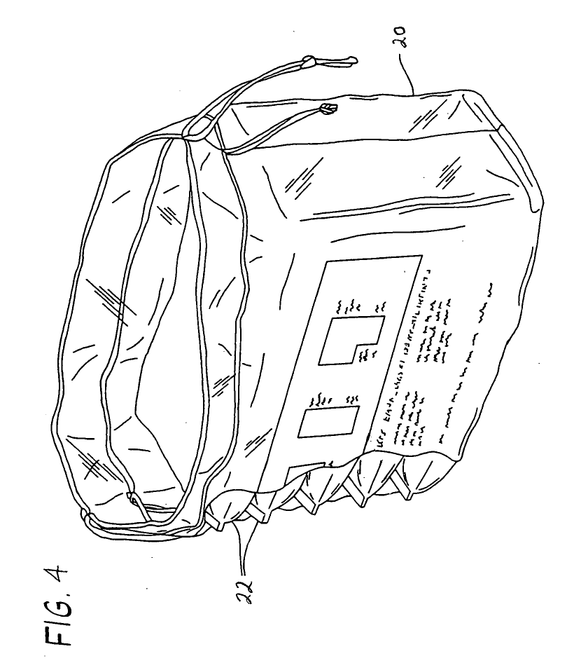

With reference to FIG. 4, the method of the invention can further include a holding of a plurality of the personal care articles 22 in a package 20. Whether wrapped or unwrapped, the individual personal care article 22 can be grouped with several other personal care articles and the grouped personal care articles can be placed in a retail container or package 20, such as provided by soft or hard packaging material. Soft packaging includes flexible envelopes and packages made of sheet plastic and/or paper. Hard packaging includes generally less flexible packages made of plastic (e.g., tubs and buckets), metal or cardboard, as well as combinations thereof. In a particular aspect, the package 20 can be a reclosable package.

As representatively shown, the package 20 can include instructions or other supplemental information which are related to the use of the articles 22. The information can, for example include text and/or graphics, as desired. Additionally, the package 20 may include information, which is related to a designation of a source of the product (e.g., a trademark or trade name). Although the various items of information may be positioned on or in the package 20 in any conventional way without departing from the scope of the present invention, a desired arrangement can have the supplemental information printed on an exterior surface of the package.

It may be desirable to package several absorbent articles 22 in a conveniently sized package (not shown) for daily use. Further, it may be desirable to package absorbent articles 22 of different types, different sizes and/or or of different absorbent capacities together in a single package. It can be also envisioned to package absorbent articles 22 having different redemption certificates, different types of redemption certificates, and/or no redemption certificates, all in a single package.

In the various arrangements of the invention, the package 20 can have any operative configuration. For example, the package 20 can comprise a bag, a carton, a container, a box or the like, as well as combinations thereof. The various panels of the package can be flexible panels, rigid panels, semi-rigid panels or the like, as well as combinations thereof. Additionally, the various panels can include any operative construction material.

As will be appreciated by those skilled in the art, the absorbent article 22 and packaging system described above may be used to more effectively establish and nurture a business relationship between a product seller and a consumer. To help establish and nurture this relationship, the product manufacturer, distributor or seller can attach a removable label or other removable layer to the product. In one embodiment, the removable layer can be separated from the product prior to using the product to enable a full functionality of the product, as described above. For example, the removable layer may be a peel-strip that covers an adhesive coating applied to the article for attaching the article to a preselected surface, or may be an individual wrapper that contains an individual article. In the various configurations of the invention, the redemption certificate can be operatively positioned on the removable layer. The redemption certificate can be directed to communicating a selected message to the user related to the acquisition of an auxiliary benefit. Additionally, the redemption certificate can be directed to communicating a selected message that pertains to motivating the user, entertaining the user, educating the user, and/or inspiring the user.

In view of the present disclosure, it is readily apparent that the several objects of the invention are achieved and other advantageous results are attained. Since various changes and modifications can be made in the above constructions without departing from the scope of the invention, it is intended that all matter contained in the above description or shown in the accompanying drawings shall be interpreted as illustrative and not in a limiting sense.

1. A method, comprising:
providing a personal care article, which includes a liquid-permeable topsheet layer, a backsheet layer operatively connected with the topsheet layer, and a removable layer which is releasably connected to the personal care article;
providing a redemption certificate on the removable layer; and
providing conversion instructions for employing the redemption certificate to obtain a separately provided item of perceived, additional benefit.

2. A method as recited in claim 1, wherein the item of benefit is provided separate from the personal care article.

3. A method as recited in claim 1, wherein the article further includes a garment-attachment mechanism operatively connected to a garment-side of the backsheet layer, and the removable layer is a peel-strip layer that has been operatively attached in a storage position to overlie and cover the garment-attachment mechanism.

4. A method as recited in claim 1, wherein the removable layer is a wrapper layer that at least partially encloses the personal care article.

5. A method as recited in claim 1, wherein the personal care article further includes an absorbent body operatively sandwiched between the topsheet layer and backsheet layer, the absorbent body capable of operatively absorbing a hygienically effective amount of urine or menses.

6. A method as recited in claim 1, wherein the redemption certificate includes a coupon redeemable for a predetermined monetary amount.

7. A method as recited in claim 1, wherein the redemption certificate includes an authentication code.

8. A method as recited in claim 1, wherein the redemption certificate includes an authentication code that is machine-readable.

9. A method as recited in claim 1, wherein the redemption certificate is configured to be substantially, non-recognizable to ordinary human vision when the removable layer is in its storage position, and is configured to become operatively recognizable to ordinary human vision upon a removal of the removable layer from the article.

10. A method as recited in claim 9, wherein
the article includes a garment-attachment mechanism configured to secure the article to an outer garment;
the removable layer is a peel-strip layer that covers the garment-attachment mechanism; and
the redemption certificate is located on a bodyside surface of the peel-strip layer.

11. A method as recited in claim 9, wherein the redemption certificate is configured to change to become operatively recognizable to ordinary human vision by employing a pattern of perforations.

12. A method as recited in claim 1, which further includes providing instructions to initiate an interactive contact with a benefit provider.

13. A method as recited in claim 1, which further includes authorizing an establishment of an interactive communication with a person seeking to redeem or actualize the redemption certificate.

14. A method as recited in claim 13, wherein the authorizing has been operatively provided by a producer of the personal care article.

15. A method as recited in claim 13, wherein the authorizing has been operatively provided by at least one intermediate entity that has been designated/appointed by the producer of the personal care article.

16. A method as recited in claim 1, which further includes authorizing a providing of a coupon in response to a receipt of the authentication code.

17. A method as recited in claim 1, which further includes authorizing a providing of a game-entry in response to a receipt of the authentication code.

18. A method as recited in claim 1, which further includes authorizing a collection and/or verification of information in response to a receipt of the authentication code.

19. A method as recited in claim 1, which further includes authorizing the presentation of an interactive, multi-pictorial graphical display in response to a receipt of the authentication code.

20. A method as recited in claim 1, which further includes authorizing the providing of a consumer-loyalty benefit in response to a receipt of the authentication code.
